(19) United States
(12) Patent Application Publication
(10) Pub. No.: US 2017/0367902 A1
(43) Pub. Date: Dec. 28, 2017

(54) MALE ABSORBENT WRAP

(71) Applicants: Joseph Thomas Paul Loder, Oshawa (CA); Sean Joseph Peter Loder, Oshawa (CA)

(72) Inventors: Joseph Thomas Paul Loder, Oshawa (CA); Sean Joseph Peter Loder, Oshawa (CA)

(21) Appl. No.: 15/631,057

(22) Filed: Jun. 23, 2017

Related U.S. Application Data
(60) Provisional application No. 62/353,657, filed on Jun. 23, 2016.

(51) Int. Cl. A61F 13/471; A61F 13/15; A61F 13/58

(52) U.S. Cl. CPC A61F 13/471; A61F 13/15203; A61F 2013/15097; A61F 13/581

(57) ABSTRACT

Described is a male absorbent wrap for wrapping around a glans. The male absorbent wrap includes a covering sheet comprising a front surface, a back surface and a first edge. The male absorbent wrap further includes a removing mechanism secured to the covering sheet proximate to the first edge and comprising a graspable portion extending away from the covering sheet.

### FIELD OF THE INVENTION

The present specification relates generally to absorbent wraps and more specifically relates to a single use, disposable wrap that absorbs body moisture in the male genital region.

### BACKGROUND OF THE INVENTION

Personal hygiene is important in maintaining good health and well-being and preventing the development and spread of infection and disease. Poor personal hygiene can cause health-related issues and have negative social effects. Men and women have unique hygiene needs and one area of specific concern is genital hygiene. For men, penile hygiene is of utmost importance, especially for intact males.

Over the past decade, routine neonatal circumcision has been declining in prevalence in the United States as health guidelines are transitioning from recommending circumcision for health reasons to making it a matter of personal choice. Therefore, the intact male population is on the rise, resulting in penile hygiene to be a growing and relevant concern.

One essential element of penile hygiene for intact males is cleanliness of the glans. During daily routine activities and more prominently during laborious activities, it is common for men to sweat in the genital area. For intact males, moisture can build up under the prepuce, causing odor and increasing the risk of infection and disease. Built up moisture can be alleviated by frequent daily cleaning of the area; however, this is not feasible and thus not common practice.

Accordingly, there remains a need for improvements in the art.

### SUMMARY OF THE INVENTION

In accordance with an aspect of the invention, there is provided a single use, disposable wrap that absorbs moisture from a male genital region.

According to an embodiment of the invention, the present invention provides a male absorbent wrap for wrapping around a glans, comprising: a covering sheet comprising a front surface, a back surface and a first edge; and a removing mechanism secured to the covering sheet proximate to the first edge and comprising a graspable portion extending away from the covering sheet.

Other aspects and features according to the present application will become apparent to those ordinarily skilled in the art upon review of the following description of embodiments of the invention in conjunction with the accompanying figures.

### BRIEF DESCRIPTION OF THE DRAWINGS

Reference will now be made to the accompanying drawings which show, by way of example only, embodiments of the invention, and how they may be carried into effect, and in which:

FIG. 1 is a front view of a male absorbent wrap according to an embodiment;

FIG. 2 is a back view of a male absorbent wrap according to an embodiment;

FIG. 3a is a male absorbent wrap in use according to an embodiment; and

FIG. 3b is a male absorbent wrap in use according to an embodiment.

Like reference numerals indicated like or corresponding elements in the drawings.

### DETAILED DESCRIPTION OF THE EMBODIMENTS

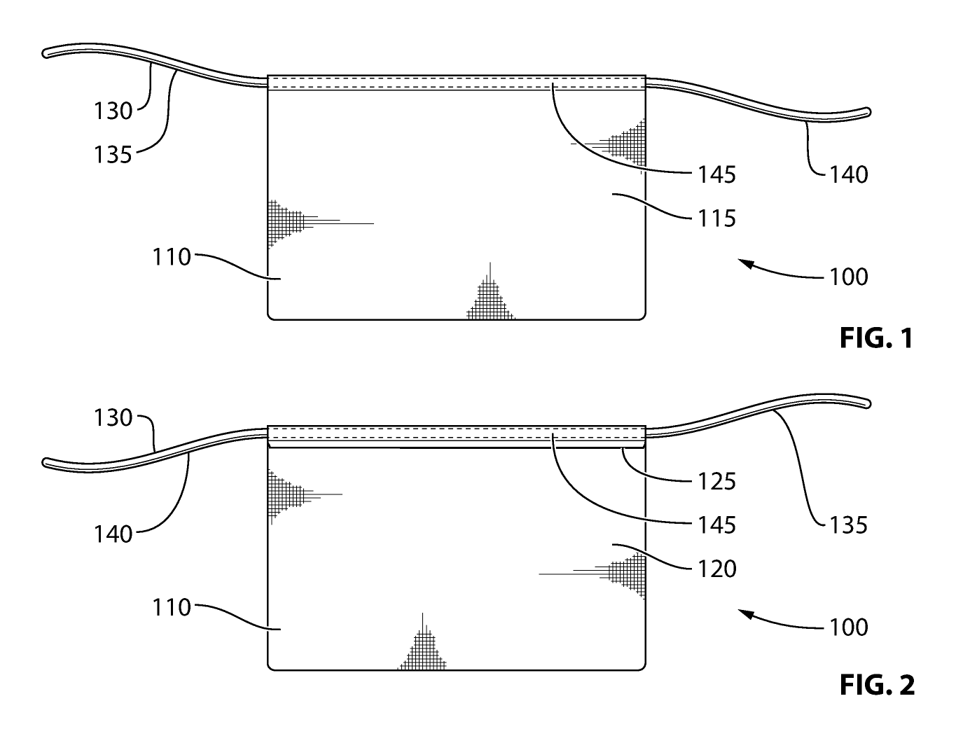

According to an embodiment as shown in FIGS. 1-2, a male absorbent wrap such as male absorbent wrap 100 is generally configured to absorb moisture from a user's, namely a male human's, genital region, particularly near the glans. The male absorbent wrap 100 may comprise a covering sheet 110, having a front surface 115, a back surface 120 and a first edge 125. The male absorbent wrap 100 may also comprise a removing mechanism, such as string 130, which may be secured to the covering sheet 110 proximate to the first edge 125 and which may comprise a graspable portion extending away from the covering sheet 110. The string 130 may have a first end 135, a second end 140 and a center section 145. The center section 145 of the string 130 may be secured to the back surface 120 of the sheet 110 proximate to the first edge 125 of the sheet 110 by folding the first edge 125 of the sheet 110 over the center section 145 of the string 130. The first edge 125 of the sheet 110 may be sewn or glued to the back surface 120 of the sheet 110 to secure the string 130. Alternatively, the center section 145 of the string 130 may be secured to the back surface 120 of the sheet 110 proximate to the first edge 125 of the sheet 110 by stitching or gluing the center section 145 of the string 130 to the back surface 120 of the sheet 110 without folding the first edge 125 of the sheet 110 over the center section 145 of the string 130.

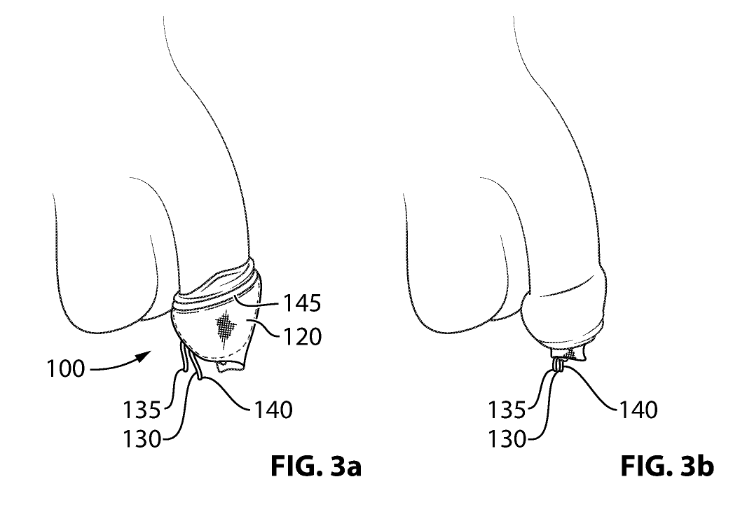

According to an embodiment as shown in FIGS. 3a-3b, the male absorbent wrap 100 may be wrapped around the user's glans, such that the string 130 wraps around the neck of the user's glans and the user's prepuce then secures the sheet 110 and the string 130 in place. The male absorbent wrap 100 may be wrapped such that the ends of the sheet 110 may slightly overlap. The sheet 110 may be in the shape of a rectangle, square or laid-flat frustoconical, such that the sheet 110 may take the shape of a cylinder or truncated cone when wrapped around the user's glans and secured by the user's prepuce.

According to an embodiment, the sheet 110 may be of such a dimension that when the male absorbent wrap 100 is wrapped around the user's glans and secured by the user's prepuce, at least a portion of the sheet 110 extends past the height of the glans. The size of the sheet 110 and the length of the string 130 may differ according to different embodiments of the invention in order to accommodate users with different glans sizes. The length of the string 130, which may be used as a removal mechanism, may be of such a length that when the male absorbent wrap 100 is wrapped around the user's glans and secured by the user's prepuce, at least a portion of the first end 135 and the second end 140 of the string 130 extend past the height of the glans. The portion of the first end 135 and the second end 140 of the string 130 that may extend past the height of the glans may be used to remove the male absorbent wrap 100 from the glans once the male absorbent wrap 100 is unsecured from the user's glans. The size of the sheet 110 may be at least 4 cm in length and 4 cm in width and the length of the string 130 may be at least 16 cm. For example, one particular embodiment of the male absorbent wrap 100 may be a rectangular sheet of 6.4 cm long by 5.1 cm wide and a string of 20 cm long, while another embodiment may be a rectangular sheet of 7.6 cm long by 5.1 cm wide and a string of 21 cm. Other dimensions are possible according to further embodiments of the invention.

According to a further embodiment, the sheet 110 may be composed of an absorbent material such as flannel, cotton, bamboo or any other disposable material capable of absorbing moisture excreted from the pores of the glans. Further, the sheet 110 may be composed of an absorbent material that aids in reducing odor. The string 130 may be composed of braided cotton, braided polyester, waxed cotton, hemp or any other material that may be used to remove the male absorbent wrap 100 from the glans.

Embodiments of the present invention as described may be suitable for use by laborers, construction workers, general contractors, landscapers, carpenters, athletes, or any male who may require absorption of moisture excreted from the glans.

The present invention may be embodied in other specific forms without departing from the spirit or essential characteristics thereof. Certain adaptations and modifications of the invention will be obvious to those skilled in the art. Therefore, the presently discussed embodiments are considered to be illustrative and not restrictive, the scope of the invention being indicated by the appended claims rather than the foregoing description and all changes which come within the meaning and range of equivalency of the claims are therefore intended to be embraced therein.

What is claimed is:

1. A male absorbent wrap for wrapping around a glans, comprising:
a covering sheet comprising a front surface, a back surface and a first edge; and
a removing mechanism secured to the covering sheet proximate to the first edge and comprising a graspable portion extending away from the covering sheet.

2. The male absorbent wrap of claim 1, wherein the covering sheet is made of an absorbent material.

3. The male absorbent wrap of claim 2, wherein the shape of the covering sheet is one of: a rectangle, square and laid-flat frustoconical.

4. The male absorbent wrap of claim 1, wherein the removing mechanism is a string composed of one of: braided cotton, braided polyester, waxed cotton and hemp.

5. The male absorbent wrap of claim 3, wherein the covering sheet is dimensioned such that, when the wrap is wrapped around the glans and secured by prepuce, at least a portion of the covering sheet extends past the height of the glans.

6. The male absorbent wrap of claim 4, wherein the string comprises a first end, a second end and a center section.

7. The male absorbent wrap of claim 6, wherein the first end and the second are graspable.

8. The male absorbent wrap of claim 6, wherein the center section of the string is secured to the back surface of the covering sheet proximate to the first edge of the covering sheet.

9. The male absorbent wrap of claim 8, wherein the first edge of the covering sheet is folded over the center section of the string onto the back surface of the covering sheet to secure the string.

10. The male absorbent wrap of claim 9, wherein the first edge of the covering sheet is sewn or glued to the back surface of the covering sheet.

11. The male absorbent wrap of claim 8, wherein the center section of the string is sewn or glued to the back surface of the covering sheet.

12. The male absorbent wrap of claim 2, wherein the absorbent material is one of: flannel, cotton and bamboo.

13. The male absorbent wrap of claim 5, wherein the covering sheet is at least 4 cm in length and 4 cm in width.

14. The male absorbent wrap of claim 1, wherein the wrap is disposable.

15. The male absorbent wrap of claim 6, wherein the string is dimensioned such that, when the wrap is wrapped around the glans and secured by prepuce, at least a portion of the first end and the second end of the string extend past the height of the glans.

16. The male absorbent wrap of claim 15, wherein the string is at least 16 cm in length.
